(19) United States
(12) Patent Application Publication
(10) Pub. No.: US 2023/0293561 A1
(43) Pub. Date: Sep. 21, 2023

(54) METHOD OF PREVENTING, REDUCING OR DELAYING FATTY LIVER DISEASE

(71) Applicant: ABBOTT LABORATORIES, Abbott Park, IL (US)

(72) Inventors: Manuel MANZANO, Granada (ES); Enrique HERNANDEZ, Granada (ES); José María LÓPEZ PEDROSA, Cenes de la Vega (ES); Ricardo RUEDA CABRERA, Granada (ES); Rachael BUCK, Gahanna, OH (US)

(21) Appl. No.: 18/006,802

(22) PCT Filed: Jul. 26, 2021
(86) PCT No.: PCT/US2021/043228

§ 371 Date: Jan. 25, 2023

Related U.S. Application Data
(60) Provisional application No. 63/059,264, filed on Jul. 31, 2020.

(51) Int. Cl. A61K 31/702; A61K 45/06; A61P 3/06; A61K 9/00; A61P 1/16

(52) U.S. Cl. CPC A61K 31/702; A61K 45/06; A61P 3/06; A61K 9/0053; A61P 1/16

(57) ABSTRACT

A method of reducing or delaying the onset of fatty liver disease in a subject comprises enterally administering at least one human milk oligosaccharide (HMO) to a subject in need thereof in an amount effective to reduce hepatic lipid accumulation. In a specific embodiment, the at least one HMO is administered in an amount effective to reduce de novo lipogenesis in the subject. The at least one HMO can be administered to the subject directly or in a nutritional composition.

### FIELD OF THE INVENTION

The present invention relates to methods of reducing or delaying the onset of fatty liver disease in a subject by administering human milk oligosaccharides (HMOs).

### BACKGROUND OF THE INVENTION

The incidence of metabolic diseases has increased significantly worldwide in the last few decades, in large part as a result of sedentary lifestyles and unhealthy eating patterns. Non-alcoholic fatty liver disease (NAFLD), which has been linked to obesity and type 2 diabetes, has been predicted to be a global epidemic. NAFLD represents a wide spectrum of diseases that originate with excess accumulation of fat within the liver, or hepatic steatosis, and is associated with multiple detrimental effects, including increased mortality due to liver failure, cardiovascular disease, and hepatocellular carcinoma. Contos M J, Sanyal A J. The Clinicopathologic Spectrum and Management of Nonalcoholic Fatty Liver Disease. Adv Anat Pathol 2002; 9(1): 37-51.

NAFLD, which is currently considered as a component of the metabolic syndrome, is an increasingly common health concern in both children and adults. In fact, it has been reported that NAFLD is the most common liver disease in the world, affecting up to one fourth of the population. See Ipsen D H, Lykkesfeldt J, Tveden-Nyborg P. Molecular mechanisms of hepatic lipid accumulation in non-alcoholic fatty liver disease. Cell Mol Life Sci 2018; 75(18): 3313-3327; Preiss D, Sattar N. Non-alcoholic fatty liver disease: an overview of prevalence, diagnosis, pathogenesis and treatment considerations. Clin Sci (Lond) 2008; 115(5): 141-150; Yu E L, Golshan S, Harlow I C E, Angeles J E, Durelle J, Goyal N P et al. Prevalence of nonalcoholic fatty liver disease in children with obesity. Pediatr 2019; 207: 64-70. Regional prevalence rates are highest in the Middle East and South America and lowest in Africa, and the incidence of NAFLD among severely obese and type 2 diabetic patients has been estimated at about 90% and 75%, respectively. Remarkably, even lean and otherwise healthy individuals have been reported to develop NAFLD. In fact, the prevalence of NAFLD has made NAFLD the second most common cause of liver transplantation in the United States. See Ipsen D H et al.; Mikolasevic, Ivana, et al. “Nonalcoholic Fatty Liver Disease and Liver Transplantation—Where Do We Stand?” World Journal of Gastroenterology, vol. 24, no. 14, 2018, pp. 1491-1506.

Unfortunately, there is currently neither an NAFLD-specific therapeutic agent available on the market nor a generally accepted NAFLD treatment. To date, the most effective treatment for NAFLD consists of implementing lifestyle changes aimed at reducing weight and increasing exercise. In addition, and given that NAFLD seems to be worsened by insulin resistance, insulin sensitizers such as pioglitazone, a thiazolidinedione compound, have been used. However, the therapeutic use of drugs that restore insulin sensitivity have raised some concerns regarding increased cardiovascular risks and other undesired side-effects. There is therefore an urgent need to develop new therapeutic strategies to fight NAFLD.

Accordingly, methods of preventing, reducing, or delaying the onset of fatty liver disease, and more particularly NAFLD, are desirable.

### SUMMARY OF THE INVENTION

It is therefore an object of the invention to provide methods which prevent, reduce, or delay the onset of fatty liver disease.

In one embodiment, the invention is directed to a method of preventing, reducing, or delaying the onset of fatty liver disease in a subject, comprising enterally administering at least one human milk oligosaccharide (HMO) to a subject in need thereof in an amount effective to reduce hepatic lipid accumulation.

In a more specific embodiment, the invention is directed to a method of preventing, reducing, or delaying the onset of fatty liver disease in a subject, comprising enterally administering at least one human milk oligosaccharide (HMO) to a subject in need thereof in an amount effective to reduce hepatic lipid accumulation, wherein the at least one HMO is administered to a subject in a nutritional composition.

The methods of preventing, reducing, or delaying the onset of fatty liver disease in a subject of the present invention are advantageous in that they provide a convenient therapeutic strategy for preventing and/or combatting fatty liver disease, and more particularly NAFLD. This and additional objects and advantages of the invention will be more fully apparent in view of the following detailed description. Accordingly, consumption of HMOs is effective and can therefore be used to for preventing, reducing, or delaying the onset of fatty liver disease by decreasing hepatic lipid accumulation.

### BRIEF DESCRIPTION OF THE DRAWINGS

The drawings are illustrative of certain embodiments of the invention and exemplary in nature and are not intended to limit the invention defined by the claims, wherein:

FIG. 1 illustrates the evolution of the body weight gain of rats fed an AlN93 diet, a high fat (HF) diet, or a HF+HMO diet over a period of 4 weeks, as described in the Example.

FIG. 2 illustrates the daily caloric intake of rats fed an AlN93 diet, a HF diet, or a HF+HMO diet over a period of 4 weeks, as described in the Example.

FIG. 3 illustrates the energy expenditure (calories/day) of rats fed an AlN93 diet, a HF diet, or a HF+HMO diet over a period of 4 weeks, as described in the Example.

FIG. 4 illustrates the final body weight gain of rats fed an AlN93 diet, a HF diet, or a HF+HMO diet over a period of 4 weeks, as described in the Example.

FIG. 5 illustrates the final fat mass of rats fed an AlN93 diet, a HF diet, or a HF+HMO diet over a period of 4 weeks, as described in the Example.

FIG. 6 illustrates the final visceral fat depots of rats fed an AlN93 diet, a HF diet, or a HF+HMO diet over a period of 4 weeks, as described in the Example.

FIG. 7 illustrates the final posterior subcutaneous fat depots of rats fed an AlN93 diet, a HF diet, or a HF+HMO diet over a period of 4 weeks, as described in the Example.

FIG. 8 illustrates the final hepatic lipid content of rats fed an AlN93 diet, a HF diet, or a HF+HMO diet over a period of 4 weeks, as described in the Example.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

The nutritional compositions and the methods described in the present disclosure can comprise, consist of, or consist essentially of any of the elements and steps as described herein.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “enterally” or “enteral administration” as used herein refers to administration involving the esophagus, stomach, and small and large intestines (i.e., the gastrointestinal tract). Examples of enteral administration include oral, sublingual, and rectal administration.

The liver has a crucial role in lipid metabolism in that it is responsible for the synthesis of new fatty acids, their export to other tissues, and their utilization as energy substrates. Hepatic lipid accumulation occurs when triglyceride production and uptake into the liver exceeds clearance or removal. The liver acquires lipids through two main pathways: the uptake of circulating fatty acids (either dietary fatty acids or non-esterified fatty acids resulting from increased lipolysis of peripheral fat depots) and via de novo lipogenesis (DNL). Hepatic lipids are removed through β-oxidation in the mitochondria and through their export as very low density lipoproteins (VLDLs). As touched on above, overeating, obesity, insulin resistance and type 2 diabetes are among the conditions that result in hepatic lipid accumulation.

As indicated above, the present invention provides methods of preventing, reducing, or delaying the onset of fatty liver disease. Without wishing to be bound by any particular theory, the methods of the present invention prevent, reduce, or delay the onset of fatty liver disease by reducing hepatic lipogenesis via administration of at least one HMO to a subject in need thereof. The present inventors have surprisingly discovered that the consumption of HMOs has a direct effect on decreasing hepatic lipid accumulation, despite not causing a decrease of weight gain or affecting the distribution of accumulated fat in both visceral fat and posterior subcutaneous fat depots. Accordingly, consumption of HMOs is effective and can therefore be used for preventing, reducing, or delaying the onset of fatty liver disease, and more particularly NAFLD, by decreasing hepatic lipid accumulation.

In one embodiment, a method of preventing, reducing, or delaying the onset of fatty liver disease in a subject is provided. The method comprises administering at least one HMO to a subject in need thereof in an amount effective to reduce hepatic lipid accumulation. In a specific embodiment of the invention, the at least one HMO is administered in an amount effective to reduce de novo lipogenesis in the subject.

In a specific embodiment, the at least one HMO can be administered to a subject from about 1 to about 6 times per day or per week, or from about 1 to about 5 times per day or per week, or from about 1 to about 4 times per day or per week, or from about 1 to about 3 times per day or per week. In more specific embodiments, the at least on HMO is administered 1 or 2 times per day, for a period of one week, two weeks, or more.

In another specific embodiment, the fatty liver disease is NAFLD. In a further specific embodiment, the subject is suffering from NAFLD. Nonalcoholic fatty liver disease comprises simple fatty liver disease, which is also referred to as nonalchoholic fatty liver (NAFL) or isolated fatty liver, and nonalcoholic steatohepatitis (NASH). Simple fatty liver disease is a form of NAFLD wherein there is fat in the liver, but there is little or no inflammation or liver cell damage. NASH, which is a more severe progression of NAFLD, is characterized by hepatic inflammation, hepatocyte damage, and/or liver fibrosis, which increases with the progression of the disease and may cause cirrhosis and hepatocellular carcinoma.

NAFLD and NASH are associated with several physiological conditions, including obesity, insulin resistance, hyperglycemia (both prediabetes and type 2 diabetes), and hyperlipidemia, all of which appear to promote the deposit of fat in the liver. Thus, these conditions, along with conditions such as hypercholesterolemia, polycystic ovarian syndrome, sleep apnea, hypothyroidism, hypopituitarism, and gut microbiota dysbiosis, may be considered risk factors for developing NAFLD.

In another embodiment of the invention, the subject is suffering from a condition selected from the group consisting of obesity, insulin resistance, prediabetes, type 2 diabetes, hyperlipidemia, hypercholesterinemia, hyperlipidemia, hypercholesterinemia, hyperlipidemia, hypercholesterinemia metabolic syndrome, hepatic inflammation, liver inflammation, hypothyroidism, hypopituitarism, and combinations of two or more thereof. In a specific embodiment, the subject is suffering from liver inflammation induced by toxins and/or an infection. In a further embodiment, the subject has undergone or will undergo chemotherapy treatment, radiotherapy treatment, and/or gastrointestinal surgery.

Examples of suitable HMOs for use in the present invention may include acidic oligosaccharides, neutral oligosaccharides, n-acetylglucosylated oligosaccharides, and HMO precursors. Specific non-limiting examples of HMOs that may be included individually or in combination in the compositions of the present disclosure include: sialic acid (i.e., free sialic acid, lipid-bound sialic acid, protein-bound sialic acid); D-glucose (Glc); D-galactose (Gal); N-acetylglucosamine (GlcNAc); L-fucose (L-Fuc); D-fucose (D-Fuc); fucosyl oligosaccharides (i.e., Lacto-N-fucopentaose I; Lacto-N-fucopentaose II; 2′-fucosyllactose (2′-FL); 3′-fucosyllactose (3′-FL); difucosyllactose (DFL); Lacto-N-fucopentaose III (LNFP III/LNP III); Lacto-N-difucohexaose I (LNDFH I); and Lactodifucotetraose (LDFT)); non-fucosylated, non-sialylated oligosaccharides (i.e., Lacto-N-tetraose (LNT) and Lacto-N-neotetraose (LNnT)); sialyl oligosaccharides (i.e., 3′-Sialyl-3-fucosyllactose; Disialomonofucosyllacto-N-neohexaose; Monofucosylmonosialyllacto-N-octaose (sialyl Lea); Sialyllacto-N-fucohexaose II; Disialyllacto-N-fucopentaose II; Monofucosyldisialyllacto-N-tetraose); and sialyl fucosyl oligosaccharides (i.e., 2′-Sialyllactose; 2-Sialyllactosamine; 3′-sialyllactose (3′-SL); 3′-Sialyllactosamine; 6′-sialyllactose (6′-SL); 6′-Sialyllactosamine; Sialyllacto-N-neotetraose c; Monosialyllacto-N-hexaose; disialyllacto-N-hexaose (DSLNH); Disialyllacto-N-hexaose I; Monosialyllacto-N-neohexaose I; Monosialyllacto-N-neohexaose II; Disialyllacto-N-neohexaose; Disialyllacto-N-tetraose; Disialyllacto-N-hexaose II; Sialyllacto-N-tetraose a; Disialyllacto-N-hexaose I; and Sialyllacto-N-tetraose b). Also useful are variants in which the glucose (Glc at the reducing end is replaced by N-acetylglucosamine (e.g., 2′-fucosyl-N-acetylglucosamine (2′-FLNac) is such a variant to 2′-fucosyllactose). Other suitable examples of HMOs that may be included in the compositions of the present disclosure include lacto-N-fucopentaose V, lacto-N-hexaose (LNH), para-lacto-N-hexaose, lacto-N-neohexaose, para-lacto-N-neohexaose, monofucosyllacto-N-hexaose II, isomeric fucosylated lacto-N-hexaose (1), isomeric fucosylated lacto-N-hexaose (3), isomeric fucosylated lacto-N-hexaose (2), fucodisialyllacto-N-hexaose (FDSLNH), fucosyllacto-N-hexaose (FLNH), difucosyl-para-lacto-N-neohexaose, difucosyl-para-lacto-N-hexaose, difucosyllacto-N-hexaose (DFNH), lacto-N-neoocataose, para-lacto-N-octanose, iso-lacto-N-octaose, lacto-N-octaose, monofucosyllacto-neoocataose, monofucosyllacto-N-ocataose, difucosyllacto-N-octaose I, difucosyllacto-N-octaose II, difucosyllacto-N-neoocataose II, difucosyllacto-N-neoocataose I, lacto-N-decaose, trifucosyllacto-N-neooctaose, trifucosyllacto-N-octaose, trifucosyl-iso-lacto-N-octaose, lacto-N-difucohexaose II (LNDFH II), sialyl-lacto-N-tetraose a, sialyl-lacto-N-tetraose b, sialyl-lacto-N-tetraose c, sialyl-fucosyl-lacto-N-tetraose I, sialyl-fucosyl-lacto-N-tetraose II, and disialyl-lacto-N-tetraose, and combinations thereof.

In a specific embodiment, the at least one HMO comprises 2′-FL, 3′-FL, LNT, 3′-SL, 6′-SL, DFNH, FDSLNH, FLNH, LDFT, LNDFH I, LNDFH II, LNFP III/LNP III, LNH, LNnT, DSLNH, DFL, or a combination of two or more thereof. In a more specific embodiment, the at least one HMO comprises 2′-FL, 3′-FL, LNT, 3′-SL, 6′-SL, or a combination of two or more thereof. In an additional embodiment, the at least one HMO comprises a mixture of 2′-FL, 3′-FL, LNT, 3′-SL, and 6′-SL. In another specific embodiment, the at least one HMO comprises 2′-FL, 6′-SL, or a combination thereof.

In a specific embodiment, the at least one HMO is administered orally. In a specific embodiment, the at least one HMO is administered to the subject in a nutritional composition. In a further specific embodiment, the nutritional composition is in the form of a powder. In another specific embodiment, the nutritional composition is in the form of a liquid.

When the nutritional composition is in the form of a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

When the nutritional composition is a powder, for example, a serving size is from about 40 g to about 60 g, such as 45 g, or 48.6 g, or 50 g, to be administered as a powder or to be reconstituted in from about 1 ml to about 500 ml of liquid, such as about 225 ml, or from about 230 ml to about 245 ml.

In a specific embodiment, the nutritional composition is administered orally. By way of example, the nutritional composition can be administered to a subject from about 1 to about 6 times per day or per week, or from about 1 to about 5 times per day or per week, or from about 1 to about 4 times per day or per week, or from about 1 to about 3 times per day or per week.

In another embodiment, the nutritional composition further comprises protein, carbohydrate, and/or a fat.

A wide variety of one or more proteins, carbohydrates, and/or fats can be used in the nutritional composition of the invention. For example, the protein can include intact, hydrolyzed, and/or partially hydrolyzed protein, which can be derived from a suitable source such as milk (e.g., casein, whey), animal (e.g., meat, fish), cereal (e.g., rice, corn), vegetable (e.g., soy, pea), and combinations thereof. In a specific embodiment, the protein comprises whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hamp proteins, flax seed proteins, earthworm protein, insect protein, or combinations of two or more thereof. The protein may also include one or a mixture of amino acids (often described as free amino acids) known for use in nutritional products, and/or metabolites thereof, or a combination of one or more such amino acids and/or metabolites, with the intact, hydrolyzed, and partially hydrolyzed proteins described herein. The amino acids may be naturally occurring or synthetic amino acids. In one embodiment, one or more branched chain amino acids (leucine, isoleucine and/or valine) and/or one or more metabolites of branched chain amino acids, for example, leucic acid (also known as α-hydroxyisocaproic acid or HICA), keto isocaproate (KIC), and/or β-hydroxy-β-methylbutyrate (HMB), are included as a protein in the nutritional compositions.

The nutritional composition may comprise protein in an amount from about 1 wt % to about 30 wt % of the nutritional composition. More specifically, the protein may be present in an amount from about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises from about 1 wt % to about 5 wt % of the nutritional composition, or from about 20 wt % to about 30 wt % of the nutritional composition.

In another specific embodiment, the carbohydrate comprises human milk oligosaccharides (HMOs), maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, inulin, fructooligosaccharides, or combinations of two or more thereof.

The nutritional composition may comprise carbohydrate in an amount from about 5 wt % to about 75 wt % of the nutritional composition. More specifically, the carbohydrate may be present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

In a further embodiment, the fat comprises coconut oil, fractionated coconut oil, soy oil, corn oil, olive oil, safflower oil, medium chain triglyceride oil (MCT oil), high gamma linolenic (GLA) safflower oil, sunflower oil, palm oil, palm kernel oil, palm olein, canola oil, marine oils, fish oils, algal oils, borage oil, cottonseed oil, fungal oils, at least one omega-3 fatty acid, interesterified oils, transesterified oils, structured lipids, and combinations of two or more thereof. In a specific embodiment, the at least one omega-3 fatty acid of the composition is selected from the group consisting of eicosapentaenoic acid, docosahexaenoic acid, arachidonic acid, and alpha-linolenic acid.

The nutritional composition may comprise fat in an amount of from about 0.5 wt % to about 30 wt % of the nutritional composition. More specifically, the fat may be present in an amount from about 0.5 wt % to about 10 wt %, about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in an amount of about 40 wt % to about 70 wt %, and a source of fat in an amount of about 5 wt % to about 20 wt %, based on the weight of the nutritional composition, and, more specifically, such composition is in powder form.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1.

In a specific embodiment, the nutritional composition comprises protein, carbohydrate, fat, and one or more nutrients selected from the group consisting of vitamins, minerals, and trace minerals. Non-limiting examples of vitamins include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof. Non-limiting examples of minerals and trace minerals include calcium, phosphorus, magnesium, zinc, manganese, sodium, potassium, molybdenum, chromium, iron, copper, and/or chloride, and combinations thereof.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

In a further specific embodiment, the nutritional composition comprises about from about 0.001 to about 15 wt %, about 0.001 to about 10 wt %, about 0.001 to about 5 wt %, about 0.001 to about 1 wt %, about 0.01 to about 15 wt %, about 0.01 to about 10 wt %, about 0.01 to about 5 wt %, about 0.01 to about 1 wt %, about 0.1 to about 15 wt %, about 0.1 to about 10 wt %, about 0.1 to about 5 wt %, about 0.1 to about 1 wt %, about 1 to about 15 wt %, about 1 to about 10 wt %, about 1 to about 5 wt %, about 5 to about 15 wt %, or about 5 to about 10 wt %, of at least one, or a combination of two or more, HMOs, based on the weight of the nutritional composition.

In a specific embodiment, the nutritional composition comprises from about 0.001 to about 10 wt %, about 0.01 to about 10 wt %, about 0.1 to about 10 wt %, about 1 to about 10 wt %, 0.001 to about 5 wt %, about 0.01 to about 5 wt %, about 0.1 to about 5 wt %, or about 1 to about 5 wt %, of at least one of 2′-FL, 3′-FL, LNT, 3′-SL, 6′-SL, DFNH, FDSLNH, FLNH, LDFT, LNDFH I, LNDFH II, LNFP III/LNP III, LNH, LNnT, DSLNH, and DFL, or a combination of two or more thereof, based on the weight of the nutritional composition.

In a further specific embodiment, the nutritional compositions comprises about 0.001 to about 10 wt %, about 0.01 to about 10 wt %, about 0.1 to about 10 wt %, about 1 to about 10 wt %, 0.001 to about 5 wt %, about 0.01 to about 5 wt %, about 0.1 to about 5 wt %, or about 1 to about 5 wt % of 2′-FL, 3′-FL, LNT, 3′-SL, and 6′-SL, and or combinations of two or more thereof, based on the weight of the nutritional composition.

In a further embodiment, the subject is a human.

The following Example demonstrates various embodiments of the invention.

### EXAMPLE

An in vivo study of the effect of HMOs in a standardized rodent model of obesity.

This example demonstrates the effect of HMOs on preventing hepatic lipid accumulation by demonstrating an in vivo reduction of hepatic lipid accumulation in an animal model of diet-induced obesity.

Thirty male Sprague-Dawley weanling rats (21-25 day old) were housed in standard cages and kept under 12 hour light-12 hour dark cycles. Room temperature was maintained at about 22° C. The rats were fed a regular nutritional diet (AlN93G diet) and water ad libitum upon arrival and until the beginning of the experiment. The rats were then divided into 3 nutritional groups and assigned a particular diet selected from: AlN93M, high fat (HF) , and HF+HMO. The details of each diet are set forth in Table 1 below.

TABLE 1
Macronutrients Profile of Experimental Diets

| | AIN93G* | HF* | HF + HMO |
|---|---|---|---|
| CHO (g/100 g diet) | 64.59 | 49.42 | 49.42 |
| Sucrose (g/100 g CHO) | 14.37 | 36.07 | 36.07 |
| CornStarch (g/100 g CHO) | 54.96 | 23.50 | 23.50 |
| MD (g/100 g CHO) | 18.97 | 24.43 | 24.43 |
| HMO: 2′FL (g/100 g diet) | | | 0.625 |
| HMO: 6′SL (g/100 g diet) | | | 0.625 |
| Cellulose (g/100 g CHO) | 1170 | 16.00 | 13.48 |
| Glycemic CHO (g/100 g CHO) | 88.30 | 84.00 | 84.00 |
| Total Fiber (g/100 g CHO) | 11.70 | 16.00 | 16.00 |
| Protein (g/100 g diet) | 17.86 | 24.19 | 24.19 |
| Fat (g/100 g diet) | 7.00 | 20.00 | 20.00 |

Over a period of 4 weeks, the rats had ad libitum access to their assigned experimental diet and water was freely available. Bodyweight and food consumption were recorded twice per week. At the end of the 4 week period, the glucose response of the rats was analyzed by a meal tolerance test. The rats were orally treated with a solution containing maltodextrin at the dose of 2 g/kg body weight. Blood samples were obtained via tail vein at baseline and at 15, 30, 60, 90, 120, 180 and 210 minutes postprandial for the glucose and insulin analysis. After one week, the rats were housed in Indirect Calorimetry (IC) cages.

After 36 hours, the rats were fasted overnight and sacrificed. Blood was immediately collected for plasma and serum isolation. Different samples (e.g., fresh feces and cecum content) and tissues (e.g., liver, pancreas, small intestine, fat depots, muscles, and bone) were removed and stored at −80° C. The effect of consuming HMOs on body weight gain, overall fat mass, visceral depots, posterior subcutaneous depots, and hepatic lipid accumulation in this rat in vivo animal model was analyzed.

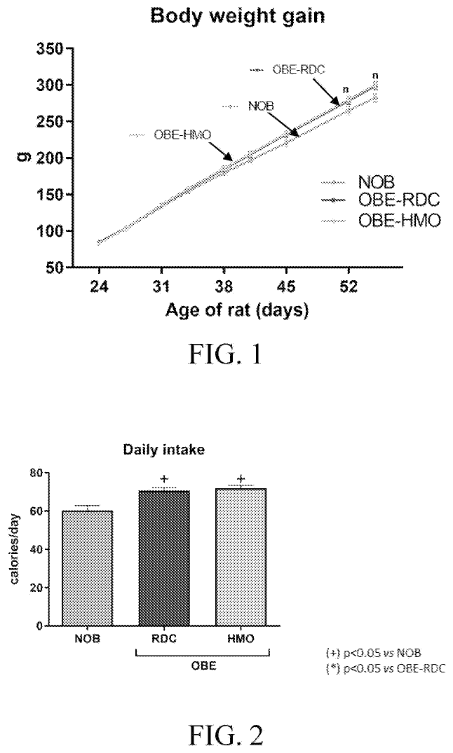

With regard to FIGS. 1-8, “NOB” refers to animals fed an AlN93M diet, “OBE” refers to both groups of animals fed a HF-based diet, “RDC” refers to animals fed a HF diet only, and “HMO” refers to animals fed a HF diet supplemented with the HMOs. As for FIGS. 2-8, the label “(+)” indicates significance versus the control (i.e., NOB) and the label “(*)” indicates significance between the HF diet only and the HF+HMO diet. A p value <0.05 is considered statistically significant.

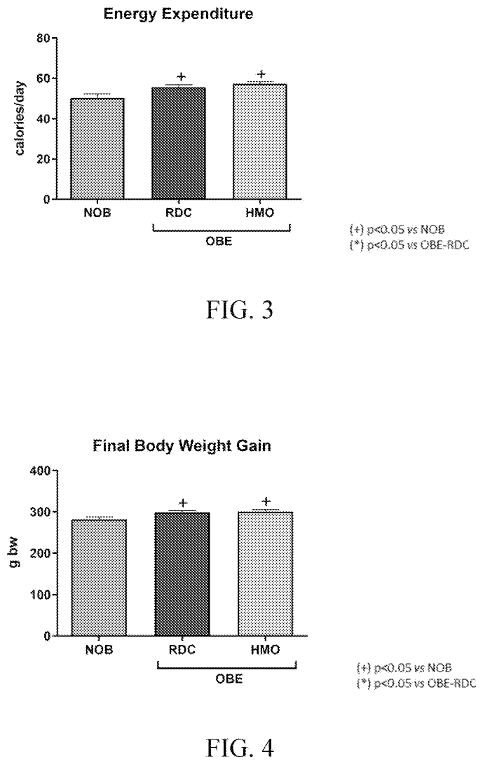

As illustrated in FIGS. 1 and 4, OBE rats that were fed the HF and HF+HMO diets, respectively, exhibited an increase in both body weight evolution (FIG. 1) and final body weight (FIG. 4), as compared to NOB rats that were fed the AlN93G diet. The results provided in FIGS. 1 and 4 thus indicate that the consumption of HMOs does not decrease total body weight gain from a high fat diet. Additionally, FIGS. 2 and 3 show OBE rats that were fed the HF and HF+HMO diets, respectively, exhibited an increase in daily caloric intake and energy expenditure in comparison with NOB rats that were fed the AlN93G diet. These results indicate that the consumption of HMOs also fails to affect feeding behavior and metabolism from consumption of a high fat diet. Overall, the results illustrate that the consumption of HMOs does not appear to be effective in preventing obesity induced by consumption of a high fat diet.

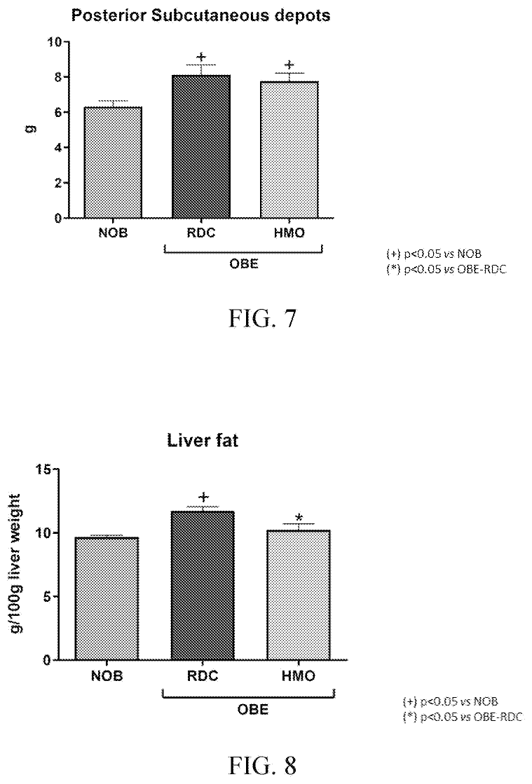

As for the effect of HMOs on visceral fat depots and posterior subcutaneous fat depots, the data provided in FIGS. 6 and 7 demonstrates that OBE rats that were fed the HF and HF+HMO diets showed a similar distribution of accumulated fat in both visceral fat and posterior subcutaneous fat depots and that the accumulated fat in these areas was higher in comparison with NOB rats that were fed the AlN93G diet. The results provided in FIGS. 6 and 7 thus indicate that the consumption of HMOs does not affect distribution of accumulated fat in both visceral fat and posterior subcutaneous fat depots.

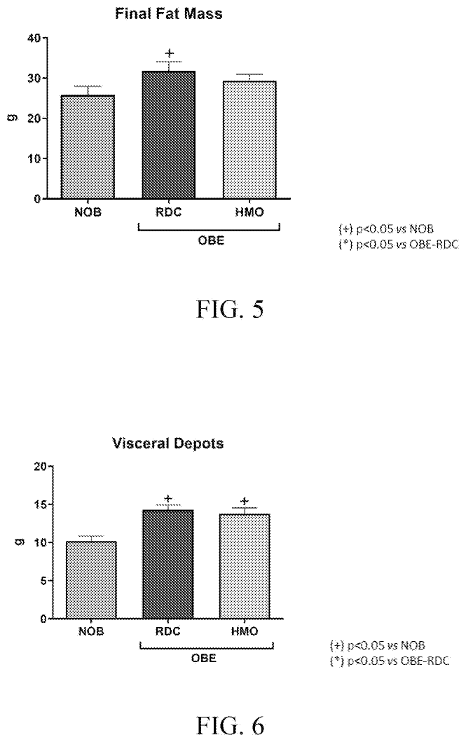

Surprisingly, however, when analyzing the total final fat mass, as set forth in FIG. 5, OBE rats that had been fed the HF+HMO diet showed a lower final fat mass in comparison with the OBE rats that had been fed the HF diet. The final fat mass of the RDC group that had been fed the HF diet was significantly higher than the NOB group. In contrast, there was no statistical difference between the HMO group as compared with the RDC group and the NOB group.

Even more surprisingly, OBE rats that had been fed the HF+HMO diet showed significantly lower hepatic lipid accumulation in comparison with the OBE rats that had been fed the HF diet, as illustrated in FIG. 8. This indicates that the consumption of HMOs has a direct effect on decreasing hepatic lipid accumulation, despite not causing a decrease of weight gain or affecting the distribution of accumulated fat in both visceral fat and posterior subcutaneous fat depots. Accordingly, consumption of HMOs is effective for preventing, reducing, or delaying the onset of fatty liver disease, particularly NAFLD, by decreasing hepatic lipid accumulation.

In summary, consuming HMOs can prevent excessive hepatic lipid accumulation, which has a significant application in preventing, reducing, or delaying diseases or conditions that are associated with increased hepatic fat content, including NAFLD.

The specific embodiments and examples described herein are exemplary only and are not limiting to the invention defined by the claims.

1. A method of preventing, reducing, or delaying the onset of fatty liver disease in a subject, comprising:
enterally administering at least one human milk oligosaccharide (HMO) to a subject in need thereof in an amount effective to reduce hepatic lipid accumulation.

2. The method of claim 1, wherein the at least one HMO is administered in an amount effective to reduce de novo lipogenesis in the subject.

3. The method of claim 1, wherein the fatty liver disease is non-alcoholic fatty liver disease (NAFLD).

4. The method of claim 3, wherein the subject is suffering from NAFLD.

5. The method of claim 3, wherein the NAFLD is simple fatty liver disease or nonalcoholic steatohepatitis (NASH).

6. The method of claim 1, wherein the subject is suffering from a condition selected from the group consisting of obesity, insulin resistance, prediabetes, type 2 diabetes, hyperlipidemia, hypercholesterinemia, hyperlipidemia, hypercholesterinemia, hyperlipidemia, hypercholesterinemia metabolic syndrome, hepatic inflammation, liver inflammation, hypothyroidism, hypopituitarism, and combinations of two or more thereof.

7. The method of claim 6, wherein the subject is suffering from liver inflammation induced by toxins and/or an infection.

8. The method of claim 1, wherein the subject has undergone or will undergo chemotherapy treatment, radiotherapy treatment, and/or gastrointestinal surgery.

9. The method of claim 1, wherein the at least one HMO comprises 2′ -fucosyllactose (2′-FL), 3′-fucosyllactose (3′-FL), lacto-N-tetraose (LNT), 3′ -sialyllactose (3′-SL), 6′-sialyllactose (6′-SL), difucosyllacto-N-hexaose (DFNH), fucodisialyllacto-N-hexaose (FDSLNH), fucosyllacto-N-hexaose (FLNH), lactodifucotetraose (LDFT), lacto-N-difucohexaose I (LNDFH I), lacto-N-difucohexaose II (LNDFH II), lacto-N-Fucopentaose III (LNFP III/LNP III), lacto-N-hexaose (LNH), lacto-N-neotetraose (LNnT), disialyllacto-N-hexaose (DSLNH), difucosyllactose (DFL), or combinations of two or more thereof.

10. The method of claim 9, wherein the at least one HMO comprises 2′-FL, 3′-FL, LNT, 3′-SL, 6′-SL, or combinations of two or more thereof.

11. The method of claim 1, wherein the HMOs are administered orally.

12. The method of claim 1, wherein the at least one HMO is administered to the subject in a nutritional composition.

13. The method of claim 12, wherein the nutritional composition is in the form of a powder.

14. The method of claim 12, wherein the nutritional composition is in the form of a liquid.

15. The method of claim 12, wherein the nutritional composition further comprises protein, carbohydrate, and/or a fat.

16. The method of claim 15, wherein the protein comprises whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hamp proteins, flax seed proteins, earthworm protein, insect protein, or combinations of two or more thereof.

17. The method of claim 15, wherein the carbohydrate comprises maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, inulin, fructooligosaccharides, or combinations of two or more thereof.

18. The method of claim 15, wherein fat comprises coconut oil, fractionated coconut oil, soy oil, corn oil, olive oil, safflower oil, medium chain triglyceride oil (MCT oil), high gamma linolenic (GLA) safflower oil, sunflower oil, palm oil, palm kernel oil, palm olein, canola oil, marine oils, fish oils, algal oils, borage oil, cottonseed oil, fungal oils, omega-3 fatty acid, interesterified oils, transesterified oils, structured lipids, or combinations of two or more thereof.

19. The method of claim 18, wherein the fat comprises at least one omega-3 fatty acid selected from the group consisting of eicosapentaenoic acid, docosahexaenoic acid, arachidonic acid, and alpha-linolenic acid.

20. The method of claim 15, wherein the nutritional composition comprises protein, carbohydrate, fat, and one or more nutrients selected from the group consisting of vitamins, minerals, and trace minerals.

21. The method of claim 15, wherein the nutritional composition comprises about 0.001 to about 10 wt % of the at least one HMO, based on the weight of the nutritional composition.

22. The method of claim 1, wherein the subject is a human.
